(19) United States
(12) Patent Application Publication
(10) Pub. No.: US 2010/0210918 A1
(43) Pub. Date: Aug. 19, 2010

(54) TASK LIGHT

(75) Inventors: David Dunn, Toronto (CA); Kevin Kowalchuk, Oakville (CA); Bryan Drew Miller, Richmond Hill (CA)

Correspondence Address: ADAMS INTELLECTUAL PROPERTY LAW, Suite 2350 Charlotte Plaza, 201 South College Street, CHARLOTTE, NC, 28244, US

(73) Assignee: JAMESON, LLC, Clover, SC (US)

(21) Appl. No.: 12/377,614

(22) PCT Filed: Aug. 23, 2007
(86) PCT No.: PCT/CA2007/001480

§ 371 Date: Apr. 28, 2010

Related U.S. Application Data
(60) Provisional application No. 60/823,504, filed on Aug. 24, 2006.

(51) Int. Cl. A61B 19/00

(52) U.S. Cl. 600/249

(57) ABSTRACT

In one aspect, the invention is directed to a treatment light comprising a support member having at least two light source support portions: each support portion adapted to be operatively connected a light source; each light source comprising one or more LEDs, particularly a plurality of LEDs associated with a focusing material which focuses the LED emitted lights into cones, the at least two light sources adapted to be fixed at spaced apart positions proximate to either side of the head of a user, the support members defining a space for positioning the head of the user next to and potentially in between the at least two light sources. Preferably, the potential contact surfaces have a steady state temperature of no greater than 120 degrees Fahrenheit when tested at an ambient temperature of 72 degrees Fahrenheit. According to one embodiment of the invention, the potential contact surfaces are no hotter than 100 degrees Fahrenheit despite generating a cumulative output of 60 to 65 watts of power.

### FIELD OF THE INVENTION

The invention relates to a task light and more particularly to an operating room light that incorporates LEDs.

### BACKGROUND OF THE INVENTION

Surgical treatment lights employed in rapidly deployable temporary field hospitals typically comprise a single incandescent or halogen light source. These medical treatment tights are typically required to withstand hot ambient temperatures of up to 130 degrees Fahrenheit and other harsh conditions. Preferred design criteria for such lights include light weight, simple operation, reduced heat emission to avoid drying living tissue or burning the user, sterile replaceable handling levers, longevity particularly reduced need for spare parts including replacement bulbs and rapid assembly and deployment into a compact easily supportable structure from a portable kit.

Typical non-portable operating room lighting comprises large multiple strong light sources which have heavy structural support systems that make them capable of adjustable positioning to avoid and fill shadows and are typically capable of wide lateral positioning to minimize shadowing attributable to the surgeons head. This type of heavy structure is impractical in rapidly deployable temporary field hospitals which provide first line care in a Forward Resuscitative Surgery System (FRSS) described herein.

### SUMMARY OF THE INVENTION

In one aspect, the invention is directed to a treatment light that is adapted for portable use by way of its relatively low weight and/or relatively small size, and wherein the light incorporates LEDs into multiple light sources which are spaced from each other.

In another aspect, the invention is directed to a treatment light that is adapted for portable use by way of its relatively low weight and/or relatively small size, and wherein the light incorporates at least two light sources which are separated by a space which can accommodate a user's head and which remains at a temperature which is less than 130 degrees Fahrenheit under steady state conditions with ambient temperature at 72 degrees Fahrenheit.

In another aspect, the invention is directed to a light with a handle mount that is changeable so that the light can accommodate a plurality of handles which have different mounting means (eg. one handle may have a particular type of thread, while another may have a different type of thread or may have a non-thread type of mounting means, such as, for example, a bayonet fitting [is this shown in the drawing?].

In another aspect, the invention is directed to a portable treatment light kit comprising a support structure having at least two light source support portions, each support portion adapted to be connected to a light source comprising a plurality of LEDs; at least two light sources each comprising a plurality of LEDs; the at least two of the light sources adapted to be readily adjusted to a position which defines at least one space between them for placement of a user's head in substantial lateral alignment therebetween when in use; each light source operatively associated with a heat elimination system capable of drawing heat away from potential contact surfaces with the user's head when positioned in the space.

In another aspect, the invention is directed to a portable treatment light that has a support structure having at least two (and in some embodiments three or more), support portions (points of attachment for light sources to be described) that are spaced apart (and in some embodiments extending or radiating from a junction in spaced apart fashion), each support portion being operatively attached to a light source having a plurality of LEDs, the light sources capable of being fixedly positioned or already pre-positioned (in virtue the spatial arrangement of the support portions to which they are attached) to define at least one space between them for placement of a user's head in lateral alignment (though not necessarily in vertical alignment) between them, when in use, each light source operatively associated with a heat transfer system for drawing heat away from the points of potential contact with the user's head when positioned in the space. By being positioned in proximity to the user's head, in approximate lateral alignment with the middle of the space and in relative close proximity to the user's head (also more closely aligned in a vertical position relative to the placement in a typical permanent operating room), the support structure itself provides a reference point to position the LED light sources so that shadowing is well reduced and the available output is well used. Using the light in this fashion is made possible by a heat reduction system that prevents burning to the touch. Importantly this combination of features has been found to be compatible with a portable lighting system, that employs LED lights which are generally longer-lasting than conventional incandescent bulbs, and is compact, rapidly assembled, easily used and rapidly adjusted.

Accordingly, in one embodiment, the invention is directed to a support structure that defines a position for the light sources relative to the head that is both adapted to avoid shadowing while also according well with a selected light focusing material and a selected distance at which the light is most needed. Optionally, this distance being somewhat longer that the distance between the user's eyes and the task surface, is within 30 to 48 inch range, optionally within the 33 to 45 inch distance range, optionally within the 36 to 42 inch range, optionally approximately one meter. The handle is optionally closely available at the center of the light sources to reposition the light to easily maintain the positioning demarcated by the positioning of the light next to the head and that accords with the heat reduction capability and the characteristics of the LED light focusing material and the watt output of the LEDs. The portable treatment light is optionally used with a flexible arm that is designed to support 15 pounds and optionally the light is therefore less than 15 pounds, optionally less than 10 pounds, optionally less than 5 pounds, optionally less than 3 pounds. The heat reduction capability is optionally selected to accord with a contact surface temperature of optionally less than 124 degrees Fahrenheit, optionally less than 120 degrees Fahrenheit, optionally less than 110 degrees Fahrenheit, optionally no greater than 100 degrees Fahrenheit. Accordingly, in a general aspect the portable treatment light of the invention has spaced LED light sources that demarcate a space for the user's head that accords with pre-selected heat dissipating and focal distance characteristics.

Accordingly, in one embodiment, the invention is directed to a portable treatment light comprising:

at least three support members radiating from a hub;

each support member supporting a light source positioned distally from the hub comprising a plurality of LED units;

each of the three support members defining at least one space between it and a respective adjacent support member for placement of a user's head between two light sources supported by two adjacent support members, when in use;

each light source operatively associated to a heat dissipater for drawing heat away from the user's head when the user's head is positioned in the space.

In another aspect, the invention is directed to a vast improvement in compact portable surgical light technology by employing long-lasting light emitting diodes as a light source.

Accordingly, in one aspect, the invention is directed to a treatment light comprising a support member having at least two light source support portions; each support portion adapted to be operatively connected a light source; each light source comprising one or more LEDs, particularly a plurality of LEDs associated with a focusing material which focuses the LED emitted lights into cones, the at least two light sources adapted to be fixed at spaced apart positions proximate to either side of the head of a user, the support members defining a space for positioning the head of the user next to and potentially in between the at least two light sources, and wherein those positions focus the respective beams of light generated by the light sources on a task surface at a distance typical of the distance between the user's head and the treatment area (approximately one meter for surgical applications in field hospitals) each light source operatively associated with a heat dissipation system capable of drawing heat away from potential contact surfaces with the head of the user when positioned in the space next to the user. Preferably, the potential contact surfaces have a steady state temperature of no greater than 120 degrees Fahrenheit when tested at an ambient temperature of 72 degrees Fahrenheit. According to one embodiment of the invention, the potential contact surfaces are no hotter than 100 degrees Fahrenheit despite generating a cumulative output of 60 to 65 watts of power. We have also found that a support member that fixes the positions of the at least two and optionally three LED-based light sources into a compact spherical area (obviating the need for a weight-adding variable positioning structure for adjusting the positions of light sources relative to one another) is able to eliminate shadows in a fashion akin to more powerful widely spaced and distantly positioned light sources without diminishing necessary illumination or generating contact surfaces that could burn the user or adversely affects the patient tissues. Accordingly we have found that focused LED light technology (including attendant advantages of the light colour variations that enhance this technology (combinations of 3500 and 5500 degree Kelvin diodes)) can be employed outside optimal permanent hospital settings (air conditioned, roomy, spacious, weight supporting, power abundant) and is compatible with the daunting rigorous demands of rapidly deployable field hospitals and other settings with comparable power, space, ambient temperature, weight supporting or portability constraints. Weight and size constraints may vary and may be set so that the task light (with support arm and base) not weigh more than 8.2 Kg (18 lbs) without its shipping case or optionally not weigh more than 16 Kg (35 lbs) in its shipping case or that the task light fit into a packing case 1220×432×87 mm (48″×17″×7″) or that any combination of these requirement be applicable. Optionally, the support arm of the task light has a range of adjustability that may include 1 m height adjustment, and/or 0.75 m radial adjustment and/or 30 degrees head angle adjustment.

Other aspects and features of the present invention will become apparent, to those ordinarily skilled in the art, upon review of the following description of the specific embodiments of the invention.

### BRIEF DESCRIPTION OF THE DRAWINGS

For a better understanding of the present invention, and to show more clearly how it may be carried into effect, reference will now be made, by way of example, to the accompanying drawings, which illustrate aspects of embodiments of the present invention and in which:

FIG. 1 is a perspective view of a task light and a user in accordance with an embodiment of the invention;

FIG. 2 is a perspective view of the task light shown in FIG. 1, shown from underneath and with some components removed for clarity;

FIG. 3 is a magnified exploded perspective view of some of the elements of the task light shown in FIG. 1;

FIG. 3a is a magnified exploded perspective view of a heat dissipation device from the task light shown in FIG. 1;

FIG. 4 is a sectional perspective view of the task light is shown in FIG. 1;

FIG. 5 is a top plan view of the task light shown in FIG. 1, illustrating a possible positioning of the head of a user; and

FIG. 6 is a plan view of a task light in accordance with another embodiment of the invention.

### DETAILED DESCRIPTION OF THE INVENTION

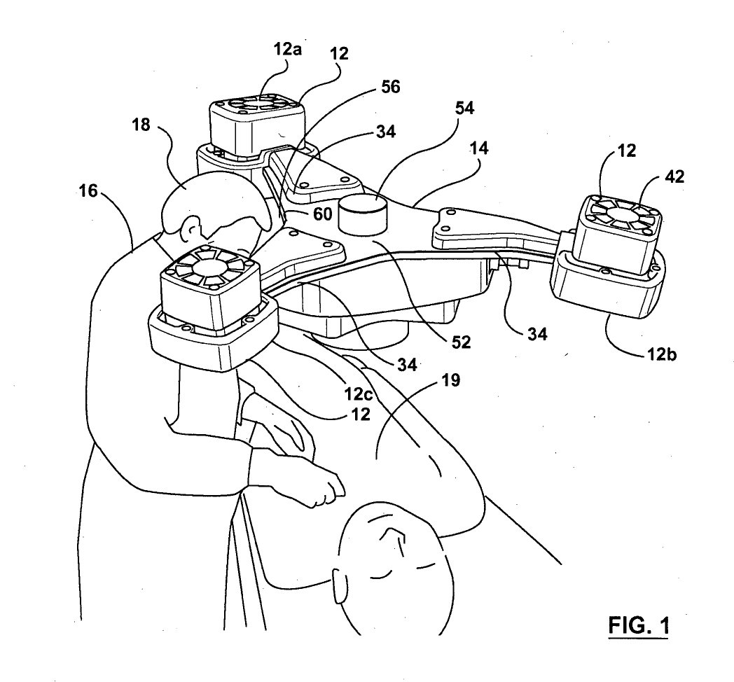

Reference is made to FIG. 1, which shows a task light 10 according to an embodiment of the present invention. The task light 10 may be used for any suitable task, such as, for example, performing a medical or dental procedure on a human patient or on an animal in an operating room, performing a medical procedure on a patient in a medical treatment facility that is portable such as one that is erected to treated injured soldiers during battle, or in performing medical examinations, or alternatively repairing a watch or other instrument with small parts.

The task light 10 includes a plurality of light sources 12, including in the exemplary embodiment shown in FIG. 1, a first light source 12a, a second light source 12b and a third light source 12c. The light 10 includes a support structure 14 which supports the light sources 12.

In one aspect, the task light 10 is advantageous in that it permits a user 16 to position it substantially at head level in a position such that a light source 12 is on one side of the head (shown at 18) of the user 16 and another light source 12 is on the other side of the head 18 of the user 16, while releasing a reduced amount of heat to the user 16 relative to some prior art lights. This position may be advantageous to the user 16 in that it permits the light 10 to be positioned dose to the work surface, shown at 19, which provides increased brightness at the work surface 19.

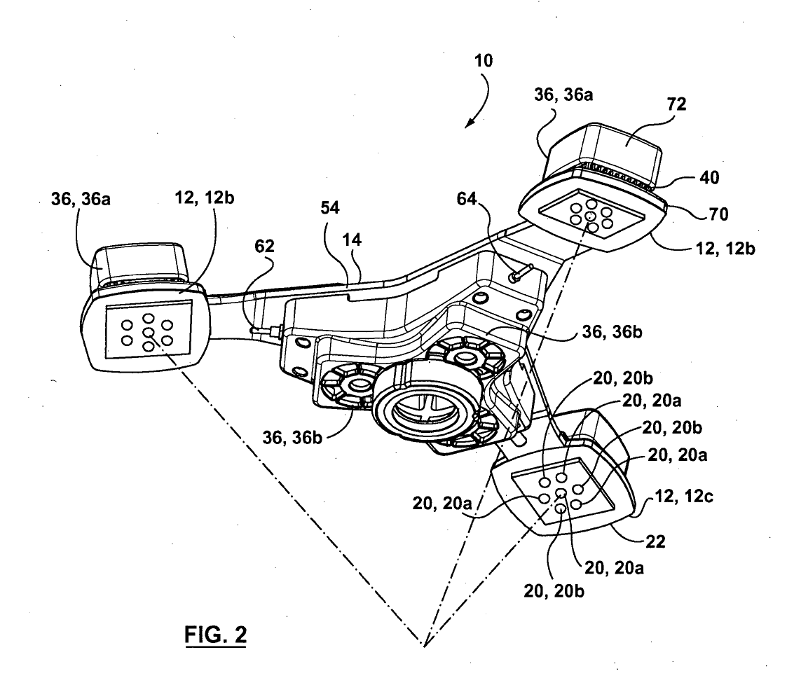

The light sources 12 may each be made up of one or more light elements 20 which may be, for example, light emitting diodes (LEDs) 20. For example, each light source 12 may contain seven LEDs 20. The LEDs 20 may be arranged in an offset pattern, which permits relatively tighter clustering, as shown in FIG. 2. For example, the LEDs 20 may be arranged in a first row of two LEDs 20, a middle or second row of three LEDs 20 in an offset relationship with the LEDs 20 in the first row and a third row of two LEDs 20 that is offset from the second row of LEDs 20, such that the seven LEDs 20 form a hexagon shaped cluster.

The LEDs 20 may include one or more first LEDs 20a and one or more second LEDs 20b. The first LEDs 20a are adapted to emit light at a first colour temperature eg. 5500 degrees Kelvin, and the second LEDs 20b are adapted to emit light at a second colour temperature, eg. 3500 degrees Kelvin. For example, in the embodiment shown in FIG. 2, each light source 12 includes four LEDs (arbitrarily referred to as first LEDs 20a) which emit light at a colour temperature of 5000 degrees Kelvin (white light) and three LEDs (arbitrarily referred to as second LEDs 20b) which emit light at 3500 degrees Kelvin (amber or yellow light).

Each light source 12 may be controlled in any suitable way. For example, the light 10 may have a main power switch 62 which controls power to the light 10 from a power source (not shown). The light 10 may further include a second LED power switch 64 which may be positionable in a first position and a second position. In the first position, the second LED power switch 64 operates the second LEDs 20b at a selected low level of power. In the second position, the second LED power switch 64 operates the second LEDs 20b at a selected low level of power. Regardless of the position of the second LED power switch 64 the one or more first LEDs 10a may operate at high power. For example, the first LEDs 20a may have a colour temperature of 5500 degrees Kelvin and the second LEDs 20b may have a colour temperature of 3500 degrees Kelvin.

The light made up of the first LEDs 20a in combination with the second LEDs 20b may have a colour temperature of approximately 5000 when the second LED power switch 64 is in the first position and a colour temperature of approximately 4300 degrees Kelvin when the second LED power switch 64 is in the second position. Other control logic may alternatively be used however instead of the aforementioned. Generally speaking the color temperature is adjusted by means of varying the pulse frequency of white and amber LEDs. Optionally, the whites may be at full power consistently and the ambers may have two settings one at full power (frequency) another which slow their pulse down (by lowering current) so that there is less amber light in the mix.

For the light 10 shown in FIG. 2 with three light sources 12, each having four first LEDs 20a and three second LEDs 20b, the overall output strength of the light 10 may be approximately 6500 lux at a distance of 1 m. Optionally, the output is a 5″ diameter spot of light optionally with a minimum intensity of 15000±2000 lux.

The output power of the light sources 12 may be expressed also in terms of wattage. Each of the LEDs 20 that make up the light sources 12 may be a 3 W LED.

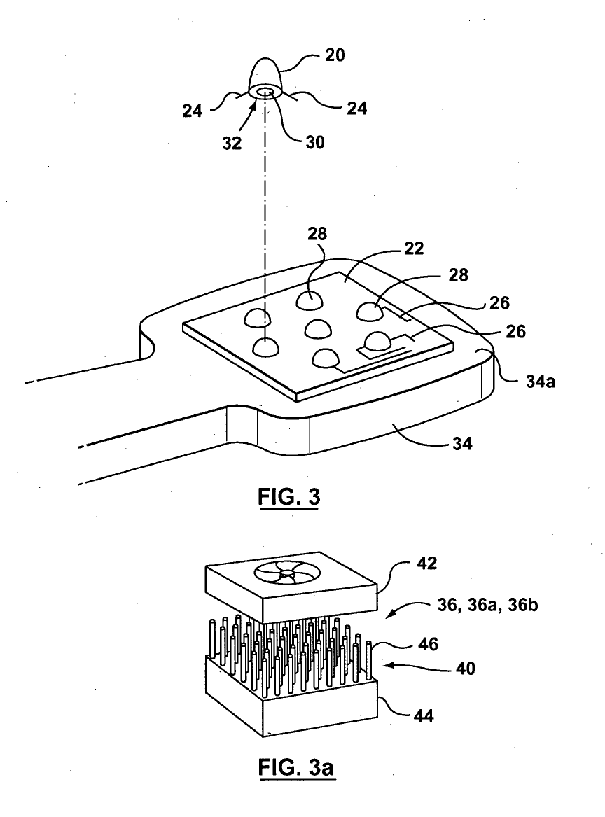

Referring to FIG. 3, the LEDs 20 may be connected to a circuit board 22 by any suitable means. For example, the LEDs 20 may each have two electrical conduits 24 which connect physically and electrically to electrical conduits 26 traced in the circuit board 22. The LEDs 20 may otherwise have no contact with the circuit board 22, and may instead pass through apertures 28 provided in the circuit board 22. Each LED 20 may have a heat conduction surface 30, which may be positioned on the aft end shown at 32. The heat conduction surface 30 may be in contact with a first end 34a of a heat transfer member 34. The heat conduction surface 30 may be made from a relatively conductive material, such as a suitable metal, to facilitate heat transfer out of the LED 20 and into the heat transfer member 34. Thermally conductive adhesive, known as thermal compound, may be used to adhere the LED 20 to the heat transfer member 34 to facilitate heat transfer therebetween. The thermal compound is preferably applied in such a way so that there are no voids therein between the heat conduction surface 30 and the heat transfer member 34. Alternatively, the LEDs 20 may directly contact the circuit board 22, which is in turn in contact with the heat transfer member via the thermal compound

The heat transfer member 34 transfers heat away from the LEDs 20 and towards a plurality of a plurality of heat dissipation devices 36 that are in thermal connection therewith. The thermal dissipation devices 36 transfer heat from the heat transfer member 34 into the environment. The heat transfer member 34 may be made from any suitably thermally conductive material such as a metallic material, such as, for example, Aluminum, which may be anodized.

The heat transfer member 34 includes a first surface 38a and an opposing second surface 38b. The first surface 38a is the surface that contacts the heat conduction surfaces 30 of the LEDs 20.

As shown in FIG. 2, the light 10 may include a first heat dissipation device 36a and a second heat dissipation device 36b that are associated with each light source 12. The heat dissipation device 36a may be positioned on the opposing second surface 38b of the heat transfer member 34 in general alignment with the set of one or more LEDs 20 in each light source 12. Thus, at least some heat is transferred from the LEDs 20 through the thickness of the heat transfer member 34 and into one of the heat dissipation devices 36a.

The heat dissipation device 36b may contact the heat transfer member 34 at a point that is spaced from the light source 12. For example, the heat dissipation device 36b may contact the heat transfer member 34 proximate a second end, shown at 34b. Thus, at least in part, heat is transferred away from the LEDs 20 along the length of the heat transfer member 34, ie. along the plane of the heat transfer member 34.

Referring to FIG. 3a, the heat dissipation device 36a may be made up of a heat sink 40 and a fan 42. The heat sink 40 may be made from a thermally conductive material, such as a metallic material, such as Aluminum, and includes a base 44 which contacts the heat transfer member 34 to draw heat therefrom, and a plurality of extensions 46 each extend outwards from the base 44 and which act to increase the surface area from which heat can escape into the environment. To increase the rate at which heat is dissipated through the extensions 46, the fan 42 is positioned to move air through the extensions 46. In this way the fan 42 causes active convection of heat from the extensions 46.

The fan 42 may be configured to draw air from the environment and to blow the air through the extensions 46 and back out to the environment. Alternatively, the fan 42 may be configured to draw air in from the environment through the extensions 46 and then through the fan itself 42 and then back out to the environment.

The heat dissipation devices 36b may be similar to the heat dissipation devices 36a, and may also each include a heat sink 48 and a fan 50. The heat sinks 48 and fans 50 may be similar in structure to the heat sinks 40 and the fans 42, however the heat sinks 48 and fans 50 may be sized to deal with the quantity of heat that reaches them via the heat transfer member 34, which may be different than the amount of heat that reaches the heat dissipations devices 36a from the light sources 12.

The heat transfer members 34 may all be integrally connected to each other. For example, they may extend outwardly from a common hub 52. As a result, the heat transfer members 34 and hub 52 are thermally connected together as part of a single integral member 54 and are therefore able to balance out to some degree any heat generation differences that might exist between the light sources 12. For example, if one of the light sources, for example 12a, generates more heat than the other heat sources, 12b and 12c in this example, or if the light source (12a in this example) is unable to dissipate heat as effectively as the others, then excess heat will be transferred through the integral member 54 towards the heat dissipation devices 36 associated with the other light sources 12. In this way, an increase in the temperature of one of the light sources 12 is at least partially dampened out by increasing the amount of heat that is dissipated by at least several of the heat dissipation devices 36.

As a result of the thermal connection between all of the heat transfer members 34, the heat dissipation devices 36b may be replaced by a single heat dissipation device, which is sized to dissipate heat transferred thereto from all of the heat transfer members 34.

To reduce the risk of damage to the LEDs 20 as a result of temperature, a thermistor may be included to sense a temperatures associated with each light source, so that the thermistor switches off its associated light source if the sensed temperature exceeds a selected limit. The thermistor may be in contact with the heat transfer member 34 proximate its first end 34a to provide temperature information regarding the light source 12 positioned at the first end 34a.

The integral heat balancing member 54 may act as the structural support 14 that supports the light sources and heat dissipation devices 36. The configuration of the integral heat balancing member 54 may be as shown in FIG. 1, including the common hub 52 and the heat transfer members 34 which act as arms that extend outwards from the common hub a selected number of degrees away from each other. For example, in the embodiment shown in FIG. 1, the heat transfer members 34 extend outwards 120 degrees apart.

By acting as a structural support and a heat transfer member, the member 54 provides two functions simultaneously and thus serves to reduce the overall weight of the device. Additionally, the shape of the member 54 is such that it provides sufficient thermal conductivity for removing heat from the light sources 12, but omits portions that would otherwise fill the spaces between the arms 34 since they do not transfer heat directly from one of the light sources 12 to one of the heat dissipation devices 36b. This further reduces the overall weight of the light 10. As a result of these and possibly other measures, the light 10 may weigh less than 3 lbs and may possibly weigh less than 2.5 lbs. As a result, the light 10 is adapted for use in portable medical care facilities, such as those facilities which are erectable in battle by the military to quickly provide care for an injured person. Such a facility is sometimes referred to as a Forward Resuscitative Surgery System (FRSS). Typically prior art lights which are used in such facilities have a single light source, which is not an LED.

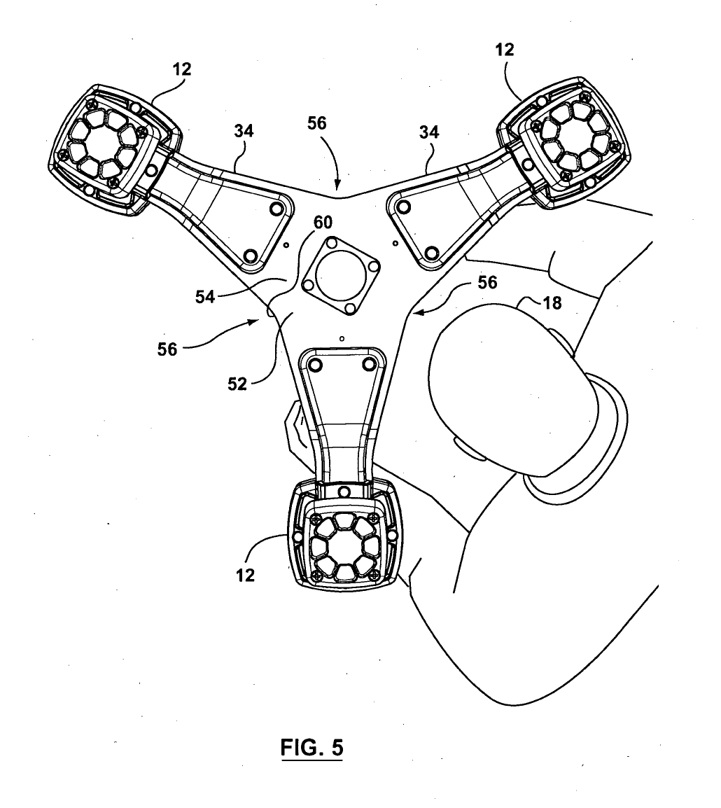

As shown in FIG. 5, a space 56 is formed between each adjacent pair of light sources 12, wherein the space 56 is sufficiently large in width (between each adjacent pair of arms 34) and in depth (radially between the light sources 12 and the hub 52) that the user 16 can position the light 10 so that one of the light sources 12 is on one side of the head 18 of the user 16 and another of the light sources 12 is on the other side of the head 18 of the user 16. For example, the width of the space 56 between housings surrounding the light sources 12 may be approximately 7.2 inches, and the depth of the space 56 may be, for example, from the outside of the LEDs 20 to the radially outer edge of the hub 52, shown at 60, may be about 1.8 inches. The horizontal distance from the outer edge 60 of the hub 52 to the centre of convergence for the light sources 12 is approximately 1.6 inches.

As a result, the user 16 can position the light 10 at head level above the work surface 19 (FIG. 1), while having light sources 12 on either side of the head 18 of the user 16. Positioning the light sources 12 at head level above the work surface 19 provides stronger illumination of the work surface 19 relative to light sources that are positioned above the head 18 of the user 16, simply as a result of the closer proximity to the work surface 19. Having light sources 12 on either side of the user 16 in combination with a light source 12 in front of the head 18 of the user 16, as shown in FIG. 1, is considered advantageous by some users who feel that it provides better illumination of the work surface 19 relative to some prior art lights with light elements that are all positioned forward of the head 18 of the user 16.

By using LEDs 20 instead of other lighting elements such as halogen lighting elements, less heat is generated at each light source 12 relative to the amount of light provided. This permits a relatively greater amount of illumination to be provided while keeping the temperature at an acceptable level for the user 16. Where ambient temperature is about 72 degrees Fahrenheit, the temperature of the housing elements 68, 70 and 72 that are shown around the light sources 12 and the first heat dissipation devices 36a can be kept below 130 degrees Fahrenheit. Optionally, the temperature of the contact surfaces of the housing elements 68, 70 and 72 can be kept below 100 degrees Fahrenheit. These temperatures apply in steady state conditions, which may occur within approximately 20 minutes of turning the light 10 on.

In addition to the relatively cool temperatures of the contact surfaces of the housings 68, 70 and 72, the light emitted by the LEDs has a relatively low component in the infra-red range and as a result, the LEDs do not emit significant quantities of heat. As a result, the tissues of the patient being illuminated are not subject to damage from drying out as a result of being illuminated by the light 10.

In addition to the heat transfer element 34 being configured to transfer heat from the light sources 12 to the heat dissipation units 36a and 36b, the heat transfer element 34 releases heat by itself into the environment. This release of heat is further assisted by having significant fraction of the surface area of the heat transfer member 34 exposed directly to the environment.

The light sources 12 may each be positioned at a selected angle with respect to the general plane of the light 10 so that their emitted light converges at a selected distance from the plane of the light 10. The plane of the light 10 is, in the exemplary embodiment shown in FIG. 1, parallel to the plane of the hub 52. The angle of the light sources 12 may be, for example, 10 degrees from the plane of the light 10. To achieve the selected angle of the light sources 12, the arms 34 may be bent by a suitable amount at a selected distance horizontally (ie. in the plane of the light 10) from the center of the light 10, such as for example, about 3.5 inches horizontally from the center of the light 10. Alternatively, in another embodiment that is not shown, the arms 34 may be co-planar with the hub 52 along their entire length and the light sources 12 may be mounted at a selected angle to the arms 34.

A light-directing element 66 may be provided which receives emitted light from the LEDs 20 and provides a selected cone angle to the emitted light. The cone angle may be, for example, 6 degrees. With this cone angle, the emitted light from the light sources 12 forms a generally circular relatively uniformly bright area on the work surface of about 8 inches in diameter, optionally about 5 inches.

The light sources 12 may be positioned at a selected radius from the centre of the light 10 so that the light coming from the three light sources 12 converges at a distance of approximately 1 m from the plane of the hub 52. For example, the light sources 12 may be positioned within a radius (or distance in embodiments wherein the light sources 12 are not positioned on a circular arc) of approximately 6.2 inches from the center of the light 10. Generally, the light sources 12 may be positioned within a radius that is within a range of about 5.2 to about 7.2 inches from the center of the light 10, while still producing a generally circular disc having a diameter of about 8 inches.

The selected distance from the plane of the light 10 at which the emitted light converges from the light sources 12 may be selected so that it corresponds generally to the distance between the level of the head 18 of a typical user 16 and the typical level of the work surface 19.

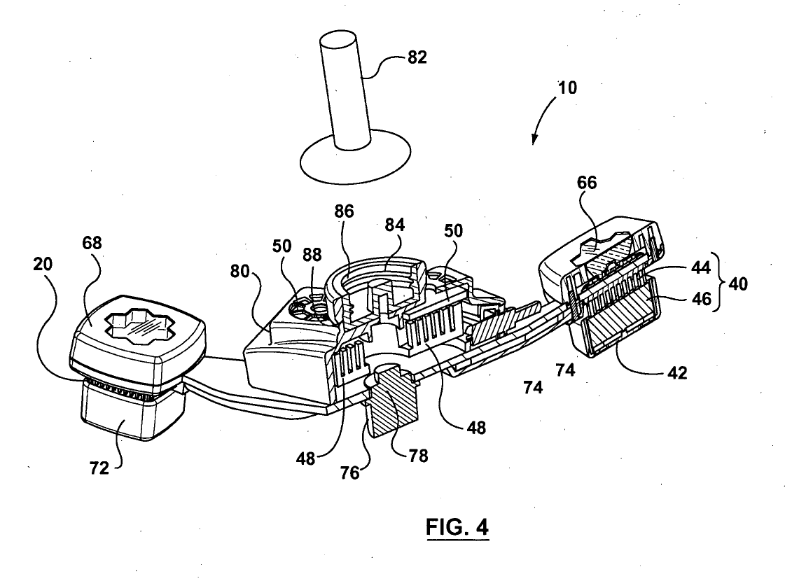

Reference is made to FIG. 4, which shows a sectional view of the light 10. The circuit board 22 may be fixed to the heat transfer member 34 by thermal compound, and may also be fixed using screws or the like. Electrical conduits shown at 74 may extend from the circuit board 22 along the heat transfer member 34 to a main circuit board 76 positioned at the hub 52. The main circuit board 76 may be responsible for conditioning incoming power for use by the LEDs 20. Electrical conduits 78 may extend from the main circuit board 76 out of the light 10 for connection to a power source. The conduit 78 may form part of the interface that connects with a flexible arm that supports the task light.

A housing 80 may be provided over the heat dissipation devices 36b, the main circuit board 76 and the switches 62 and 64. The housing 80, and the housings 68, 70 and 72 may all be made from a suitable polymeric material which is relatively thermally non-conductive.

Variations in cone angle and converging distance are contemplated.

Referring to FIG. 4, the light 10 may be configured to receive a handle 82, which may be a standard sterile handle which is in common use and which has a male thread that mates with a female thread 84 provided in a removable handle mount 86. In the event that a different handle becomes a common standard in the industry, and it has a different means of mounting to a light, the handle mount 86 may be removed and replaced with a new handle mount that is configured to receive the new handle. The handle mount 86 may be connected to the rest of the light 10 in any suitable way. For example, the handle mount 86 may be press-fit in a receiving aperture 88 in the housing 80.

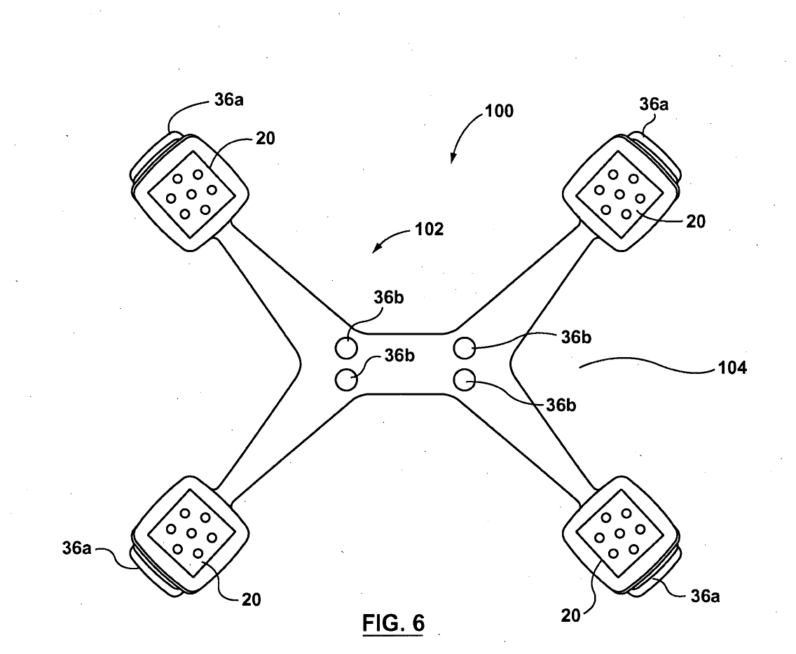

Reference is made to FIG. 6, which illustrates a light 100 in accordance with a second embodiment of the present invention. The light 100 may be similar to the light 10 (FIG. 1), except that the light 100 includes four light sources 12. Each light source 12 may have a heat transfer member 34 associated therewith and first and second heat dissipation devices 36a and 36b associated therewith. The spaces 102 and 104 may be provided between adjacent pairs of light sources 12, which are sufficiently large in width and depth to permit the light sources 12 to be positioned on either side of the head 18 of the user 16.

A light in accordance with an embodiment of the invention may have as few as two light sources. Alternatively it may have five or more light sources.

The term “opposite sides of the head” is used to define positions of the lights sources relative to the head of the user and is understood to mean that the support structure together with the light sources define a notch-like space for head placement that is large and deep enough for the user to position his/her head between the light sources to an extent that the light sources are proximate to the respective coronal sutures on either side of the head.

While the above description provides example embodiments, it will be appreciated that the present invention is susceptible to modification and change without departing from the fair meaning and scope of the accompanying claims. Accordingly, what has been described is merely illustrative of the application of aspects of embodiments of the invention. Numerous modifications and variations of the present invention are possible in light of the above teachings. It is therefore to be understood that within the scope of the appended claims, the invention may be practiced otherwise than as specifically described herein.

1. A portable treatment light comprising:
(a) a support member having at least two light source support portions, each support portion adapted to be operatively connected to a light source;
(b) each light source comprising a plurality of LEDs associated with a focusing material which focuses each LED emitted light into a cone of light of about at least 10 degrees;
(c) the at least two light sources adapted to be fixed at predetermined spaced apart positions on opposite sides of the head of a user, and wherein the focus of the respective beams of light generated by the light sources is directed onto a task surface at a distance of approximately one meter;
(d) each light source operatively associated with a heat dissipation assembly capable of drawing heat away from potential contract surfaces sufficient to maintain a contact surface temperature at a steady state temperature of no greater than 136 degrees Fahrenheit when tested at an ambient temperature of 72 degrees Fahrenheit.

2. The portable treatment light of claim 1, wherein the support member comprises at least three support portions radiating from a junction.

3. The portable treatment light of claim 1, wherein the support member comprises at least three support arms radiating from a central junction.

4. (canceled)

5. (canceled)

6. The portable treatment light according to claim 1 wherein the support arms radiate from a central junction at equal angles to form at least three equidistantly spaced arms.

7. The portable treatment light of claim 1, wherein the support member is made from a thermally conductive material and wherein the support member is in thermal contact with the plurality of LEDs to conduct heat away from the LEDs.

8. (canceled)

9. The portable treatment light of claim 1, wherein at least one heat dissipation assembly is in thermal contact with the support member at a position that is spaced from the light sources, wherein the at least one heat dissipation assembly is configured to draw heat from the support member and dissipate the heat into the ambient environment.

10. The portable treatment light of claim 1, including a focusing material that focuses the LED emitted light into a cone of 6 degrees.

11. A portable treatment light according to claim 3, wherein the central junction is a hub, the junction of the support arms to the hub defining a reference plane and wherein at least a distal portion of the support arm radiates at an angle relative to the reference plane in the direction of a plane of the task surface.

12.-19. (canceled)

20. A portable treatment light kit comprising:
(a) a support structure having at least two light source support portions, each support portion adapted to be connected to a light source comprising a plurality of LEDs;
(b) at least two light sources each comprising a plurality of LEDs, the at least two light sources each adapted to be adjusted to a position which defines at least one space between them for placement of a user's head substantially laterally therebetween, when in use; and
each light source operatively associated with a heat elimination assembly capable of drawing heat away from potential contract surfaces with the user's head when positioned in the space between the at least two light sources.

21. A portable treatment light comprising:
a support structure having at least three support portions radiating from a junction, each support portion operatively attached to a light source having a plurality of LEDs, the light sources positioned to define at least one space between them for placement of a user's head between them, when in use, each light source operatively associated with a heat sink for drawing heat away from the user's head when positioned in the space between the support portions.

22. A portable light according to claim 21, and comprising:
(a) at least three support members radiating from a hub, each support member supporting a light source positioned distally from the hub comprising a plurality of LED units; and
(b) each of the three support members defining at least one space between it and a respective adjacent support member for placement of a user's head between two light sources supported by two adjacent support members, when in use.

23. (canceled)

24. A portable light according to claim 21, wherein the at least two light sources are positioned to emit light beams which converge at a point that is approximately 1 m below the plane of the light sources, and wherein the light sources are positioned generally at points on a circle having a radius of about 6″.

25.-31. (canceled)

32. A portable light according to claim 24, wherein the three support members are formed integrally with the hub.

33.-37. (canceled)

38. A portable light according to claim 21, wherein at a distance of 1 m, the light generates an approximately 5″ diameter spot of light with a minimum intensity of 15000±2000 lux.

39. A portable light according to claim 21, wherein the light has a color temperature at 3750K+/−300K for a first setting and 4500K+/−300K for a second setting.
